(19) United States
(12) Patent Application Publication
(10) Pub. No.: US 2010/0114302 A1
(43) Pub. Date: May 6, 2010

(54) ENDOVASCULAR DEVICES WITH AXIAL PERTURBATIONS

(76) Inventors: Abraham Tzafriri, Cambridge, MA (US); Kumaran Kolandaivelu, Jamaica Plain, MA (US); Elazer R. Edelman, Brookline, MA (US)

Correspondence Address: K&L Gates LLP, STATE STREET FINANCIAL CENTER, One Lincoln Street, BOSTON, MA, 02111-2950, US

(21) Appl. No.: 12/374,913

(22) PCT Filed: Jul. 24, 2007
(86) PCT No.: PCT/US07/16629

§ 371 Date: Dec. 14, 2009

Related U.S. Application Data
(60) Provisional application No. 60/832,771, filed on Jul. 24, 2006.

(51) Int. Cl. A61F 2/06

(52) U.S. Cl. 623/1.42; 623/1.1

(57) ABSTRACT

Endovascular devices are provided. The endovascular devices include a conformable scaffold with one or more outpocketings. The outpocketing in the endovascular device creates a corresponding outpocketing of a vessel wall, thereby altering local fluid dynamics.

### FIELD OF THE INVENTION

This invention relates generally to the establishment of pockets or wells at specified sites within the vascular tree for altering the local convective transport of agents to and from the vessel wall wherein those pockets are created by the placement of an endovascular device such as a stent.

### BACKGROUND

The local hemodynamic environment is a powerful modulator of vascular state. Disruption of physiological flow can lead to a variety of diseases and their pathological progression. Bounding examples of such flow disruption include cases of stenosis and aneurismal dilation. In stenotic regions, such as those that occur in cases of cumulative atherosclerotic disease or dysplastic processes, blood flow is impeded, thus preventing transport of vital substances to and from given sites such as the heart, brain, kidney, intestines, eyes, or extremities. Aneurysmal dilations create unphysiological zones of low, recirculant shear, altering the normal convective wall transport and increasing the risk of thrombotic occlusion or embolization through the pathological recruitment of innate, blood-borne agents. Diseases caused by such vascular pathologies are among the leading cause of morbidity and mortality in the Western world, and as a result, much effort has been placed in developing suitable therapies.

The highly localized, discrete nature of such vascular pathologies and their ready accessibility through existent intra-luminal technologies make them ideal targets for local therapeutic interventions which typically seek to reinstate physiological flow patterns. Angioplasty has transformed the treatment of localized vascular disease. By itself, it can treat stenotic regions through plastic, radial deformations of the vessel wall. Another major advance has been the establishment of mesh structures that can be expanded on these balloons or other modalities, thereby establishing a secure, tubular lumen through diseased stenotic or dilated segments. Newer devices, such as Y-shaped implants placed at a bifurcation, are being used to reestablish more complex vascular geometries.

These revolutionary devices have changed the way vascular diseases are managed and yet they are not without adverse consequence. Exaggerated biological responses such as smooth muscle cell overgrowth and blood clot occur on these implanted, foreign structures. A great deal of effort has been spent in trying to develop material properties that enhance biocompatibility or biodegradability in an effort to minimize the effects of device presence to those required for maintaining normal vessel architecture. However, the formulation of such ‘invisible’ devices has remained elusive and their implementation has been ridden with difficulty. Alternatively, conjugate systemic therapies which attempt to blunt untoward reactions of these devices have become traditional practice though these create problems given the potential for systemic side effects.

Recently, increasing focus has been placed on the development of local drug delivery which avoids major systemic distribution and toxicity. Catheters have been designed which inject agents directly at a desired and specific location in the vascular tree. Recently, stents have been coated with drug as a method of administering drug long after catheter retraction. Such implants provide a mechanism of treating the acute and/or chronic adverse effects associated with stent presence.

It is well recognized that the therapeutic efficacy of such local delivery strategies is governed by a variety of local pharmacokinetic transport phenomena (Hwang et al. (2003) Int. J. Cardiovasc. Interventions 5(1):7-12). As in the case of systemically administered agents, the dosing and distribution of local agents are crucial factors in maximizing therapeutic benefit while minimizing toxicity. This suggests that local drug delivery can be optimized by appropriately tailoring release kinetics and distribution profiles and accordingly a variety of technologies have been developed in an effort to control local pharmacokinetics (see, for example, U.S. Patent Application Publication No. 2003/0083646 A1 and U.S. Pat. Nos. 6,939,375; 6,858,221; 6,491,666; and 6,471,980). An intriguing strategy has made use of systemic agents which synergize with local agents to enhance local effect in an effort obtain therapeutic control and minimize systemic toxicity (see U.S. Patent Application Publication No. 2003/0083739 A1).

As stated previously, flow is known to be a powerful modulator of vascular state. The current trend in device design is to normalize flow patterns by minimizing zones of stagnation and recirculation that are implicated in the pathogenesis of device failure. It has recently been established that zones of stagnation and recirculation induced by strut architecture affect drug delivery from stents (Balakrishnan et al. (2005) Circulation 111(22):2958-65).

Current treatments for stenosed arteries generally seek to reestablish a constant luminal circumference with the flanking region, thereby promoting patency. Through the use of low profile stent or strut design, this approach generally balances adverse effects related to stent deployment and presence with the need to withstand sufficient radial stresses. Thus, flow alterations are generally limited to those associated with strut protrusion into the lumen and uncontrolled edge effects.

### SUMMARY OF THE INVENTION

The present invention permits design of an endovascular device to controllably alter vascular flow. In the pathological context of a vascular aneurysm, a localized outpouching of the vessel wall leads to altered convective transport and the buildup of bioactive, thrombotic agents. In the present invention, an endovascular device is designed to promote an outpocketing of the vessel wall, creating a flow perturbation that is useful, for example, for localizing deposition or recruitment of an endogenous or exogenous bioactive agent, providing improved control over local concentrations of a drug, antibody, enzyme, or small molecule.

Thus, in one aspect, the invention relates to an endovascular device that includes a conformable scaffold designed for insertion into a blood vessel and expansion within it. The scaffold includes one or more shaping elements that form, after expansion of the scaffold within the blood vessel, one or more outpocketings of predetermined shape in the scaffold and the wall of the blood vessel. The expanded scaffold includes a lumen permitting blood flow through the scaffold and the blood vessel. In one embodiment, at least one outpocketing creates a recirculation zone. The shaping element is formed in one embodiment of a shape-memory material such that, upon expansion of the scaffold, the shape-memory material expands to form at least one outpocketing of predetermined shape. In another embodiment, the shaping element includes struts or wires of nonuniform thickness. In various embodiments, the outpocketing is radially symmetric or radially asymmetric. A bioactive agent, such as a drug, antibody, enzyme, or small molecule, is optionally releasably associated with the scaffold or elsewhere on or in the endovascular device.

A partition partially or completely separating the outpocketing from the lumen of an endovascular device permits further regulation of flow and mass transport parameters. Accordingly, in one aspect, the invention relates to an endovascular device that includes a conformable scaffold and a partition. The conformable scaffold is designed for insertion into a blood vessel and expansion within it and includes, after expansion within the blood vessel, a lumen permitting blood flow through the scaffold and blood vessel and one or more outpocketings of predetermined shape. The partition is between the outpocketing and the lumen. The partition is not apposed to the scaffold in the outpocketing and thereby defines a space between the outpocketing and the partition. The material that forms the partition can be a material more elastic than the conformable scaffold and can form a part of the device prior to insertion and expansion within a blood vessel. In another embodiment, the material that forms the partition is inserted into the device following expansion of the scaffold and can be relatively plastic or elastic. In one embodiment, the partition forms a barrier to convective blood flow into and out of the space. In another embodiment, the partition forms a barrier to diffusion into and out of the space. In one embodiment, the device includes a bioactive agent releasably associated with the scaffold or with a surface of the partition that is not in contact with the lumen; the device optionally also includes a second bioactive agent releasably associated with a surface of the partition that is in contact with the lumen. A surface of the partition that is in contact with the lumen can include one or more molecules (such as an extracellular matrix molecule) that binds endothelial cells or endothelial precursor cells.

In another aspect, the invention relates to a method of introducing an outpocketing into a vessel wall. The method includes inserting into a blood vessel a conformable scaffold designed to deform a wall of the blood vessel. The scaffold is then radially expanded and, after expansion, includes a lumen permitting blood flow through the scaffold and blood vessel. At least one outpocketing is controllably introduced in the scaffold and the wall of the blood vessel. In one embodiment, the outpocketing is controllably introduced concurrently with the radial expansion of the scaffold. Radial expansion is optionally performed by expanding a balloon within the lumen of the scaffold, or by other means. In one embodiment, the outpocketing is created by conforming the scaffold to a specific balloon contour. In one embodiment, the outpocketing creates a recirculation zone and can be radially symmetric or asymmetric. In one embodiment, the scaffold includes a bioactive agent such as a drug, antibody, enzyme, or small molecule releasably associated with the scaffold.

In another aspect, the invention relates to a method of altering fluid flow in an endovascular device. The method includes inserting a partition into a conformable scaffold that has been expanded to be in contact with an interior wall of a blood vessel. The scaffold has a lumen permitting blood flow through the scaffold and the blood vessel. The scaffold and the interior wall of the blood vessel include at least one outpocketing. The inserted partition is positioned between the outpocketing and the lumen, but is not apposed to the scaffold in the outpocketing. The partition thereby defines a space between itself and the outpocketing. In one embodiment, the partition creates a barrier to convective blood flow into and out of the space. In another embodiment, the partition creates a barrier to diffusion into and out of the space. In a particular embodiment, a surface of the partition that includes one or more molecules that bind endothelial cells or precursor cells is positioned to be in contact with the lumen.

The method of altering fluid flow can also incorporate one or more additional steps. In one embodiment, the method includes the additional step of immobilizing the partition with respect to the endovascular device. In another embodiment, the partition, which can be plastically deformable, is expanded after it is inserted into the expanded conformable scaffold.

Additional aspects and embodiments are disclosed in the drawings and detailed description of the invention, which follow.

### BRIEF DESCRIPTION OF THE DRAWINGS

FIG. 1 is a schematic depiction of an endovascular device that does not include an outpocketing.

FIG. 2 is a schematic depiction of an endovascular device with an axially symmetric outpocketing.

FIG. 3 is a schematic depiction of an endovascular device with an axially asymmetric outpocketing.

FIG. 4 is a schematic depiction of an endovascular device non-uniformly expanded with an axially symmetric, nontubular balloon.

FIG. 5 is a schematic depiction of an endovascular device non-uniformly expanded with an axially asymmetric balloon.

FIG. 6 is a schematic depiction of an endovascular device with an outpocketing following expansion with a tubular balloon.

FIG. 7 is a schematic depiction of an endovascular device with more than one outpocketing.

FIG. 8 is a schematic depiction of an endovascular device with a fenestrated partition separating the lumen from an outpocketing.

FIG. 9 is a schematic depiction of an endovascular device with a full partition separating the lumen from an outpocketing.

### DETAILED DESCRIPTION OF THE INVENTION

The current invention modulates and optimizes local flow-based transport phenomena by manipulating the overall geometry of an endovascular device incorporating a conformable scaffold. Specifically, the controlled introduction of one or more outpocketings into the shape of the conformable scaffold and the resulting deformation of the vessel wall and lumen alter flow-based transport properties by changing the geometry of the fluid path. Generally, this is to be accomplished through the expansion of the vessel wall to a desired shape through the use of an expansion technique and the maintenance of this shape with an implanted device. The expansion technique may use a device which is different than the implanted device, where this expansion imposes a desired shape in the implanted device and vessel wall.

### Scaffolds

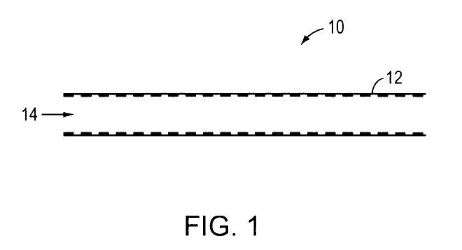

The present invention permits the modulation of endovascular flow-based phenomena through the use of an endovascular device. An exemplary endovascular device is depicted in FIG. 1. Referring to FIG. 1, endovascular device 10 incorporates conformable scaffold 12, which is engineered to be of sufficient mechanical design to support the stresses of a vessel wall. Typically, endovascular device 10 is designed to be deployed into a vessel using a catheter or other delivery mechanism that moves within the vessel and delivers the endovascular device to the appropriate endovascular location. Conformable scaffold 12 is then generally expanded within the vessel at least until the scaffold has achieved a size and shape sufficient to contact the vessel wall and to immobilize conformable scaffold 12 within the vessel. Lumen 14 passes through conformable scaffold 12, permitting fluid flow through conformable scaffold 12 and through the blood vessel.

Scaffolds preferably incorporate a plastically deformable material such that the scaffold can go from a collapsed state to an expanded state. The material is of sufficient mechanical strength to support the intended expanded deformations in the vessel wall (when incorporated into a specific scaffold). Many metals have the required mechanical properties; exemplary metals include stainless steel alloys (316L, for example), cobalt alloys (cobalt chromium, for example), titanium alloys (nitinol, for example, known for its self-expanding properties) amongst other metals such as platinum alloys, magnesium alloys, etc. Other materials such as polymers (e.g. poly-L-lactic acid) are also useful. Such materials have been widely studied and implemented in endovascular device technologies and are further notable for their biocompatibility (Poncin et al., “Stent Tubing: Understanding the Desired Attributes,” Materials & Processes for Medical Devices Conference, ASM International, 8-10 Sep. 2003). While these materials, among others, are useful in forming the structural backbone of the scaffold, the final structure can incorporate a composite such that the backbone is covered with a coating layer altering the scaffold's biocompatibility or its ability to incorporate or elute bioactive agents.

Many commonly employed methods currently directed towards endovascular stent design can be used to form scaffolds from these materials. These include, for example, chemically etched or laser cut slotted tube construction or chemically etched or laser cut flat plate design followed by rolling, bent-wire frame construction, etc. (Serruys et al. (2000) Handbook of Coronary Stents, Third Ed.; London: Martin Duntz).

### Outpocketings

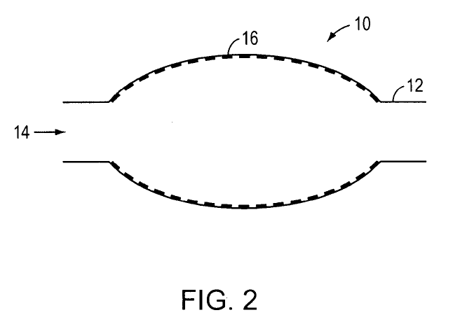

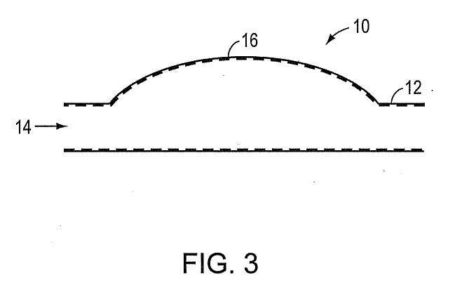

As shown in FIG. 2, one or more outpocketings 16 are controllably introduced into conformable scaffold 12 and, thereby, into the end luminal shape of the target vessel. Although outpocketing 16 of endovascular device 10 of FIG. 2 is axially symmetric, axially asymmetric outpocketings, such as outpocketing 16 of endovascular device 10 of FIG. 3, are also useful. Importantly, an outpocketing is generally not of the same geometry as the native, physiological vessel architecture, but rather is designed specifically to induce specifically altered local transport.

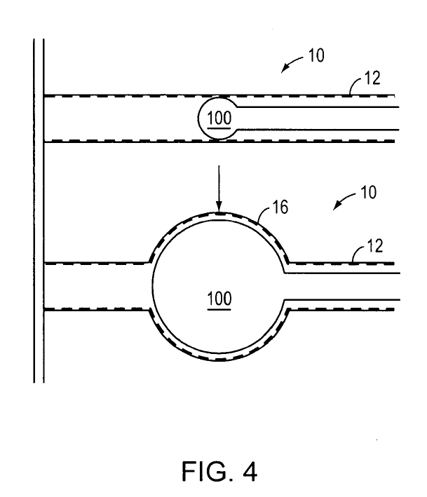

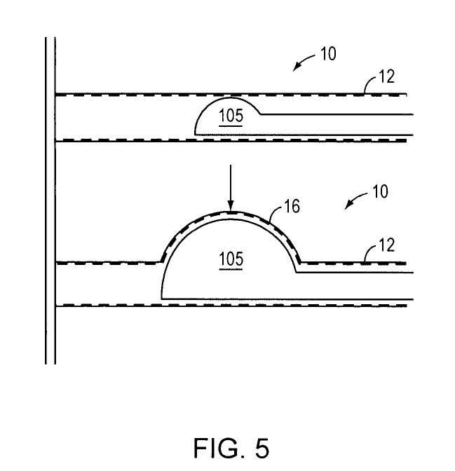

The shape of an outpocketing can be determined by the physician at the time of implantation. For example, FIG. 1 depicts an endovascular device 10 whose conformable scaffold 12 is of substantially uniform structure throughout its length, such that, if radially expanded in a uniform fashion, conformable scaffold 12 will take on a substantially tubular shape in the blood vessel. If, however, endovascular device 10 is expanded in a nonuniform fashion, as depicted in FIG. 4, conformable scaffold 12 will include one or more outpocketings 16. Expansion of the endovascular device 10 using a non-tubular balloon 100 permits the introduction of an outpocketing; the shape of the non-tubular balloon 100 defines the shape of outpocketing 16. Thus, non-tubular balloon 100 in FIG. 4 is axially symmetric and introduces axially symmetric outpocketing 16. In contrast, as depicted in FIG. 5, use of axially asymmetric non-tubular balloon 105 introduces an axially asymmetric outpocketing 16 into endovascular device 10. By selecting the balloon, its location along the axis of conformable scaffold 12, and the degree of inflation, the physician can dictate the geometry of outpocketing 16.

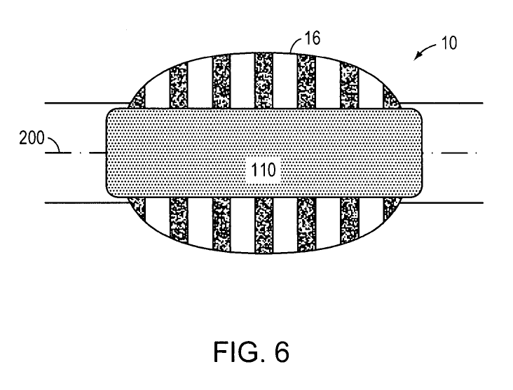

The shape of an outpocketing can also be predetermined by the properties of the endovascular device, as shown in FIG. 6. Referring to FIG. 6, implanted endovascular device 10 is expanded using an expansion device 200, which can be a balloon catheter in which balloon 110 expands to a constant, tubular diameter. In this case, the shape of outpocketing 16 is defined by properties of endovascular device 10. Examples of these properties can include struts or wires of altered geometry (e.g. depth) such that the shape of lumen 14 is cylindrical, while the outer, wall-apposed shape defines the desired end-vessel shape.

Another available shape-defining property for outpocketing 16 relies on the use of a shape-memory alloy such as nitinol. The implanted device can be expanded to a nominal shape (e.g. tubular) which is not the desired end shape. Following implantation, the device may then alter its shape to its characteristic ‘memory,’ thereby imposing the desired end shape on the vessel.

These implanted devices are of sufficient mechanical design to support the stresses of the vessel wall which, in general, will not be the same stresses as those imposed with expansion to a nominal tubular geometry.

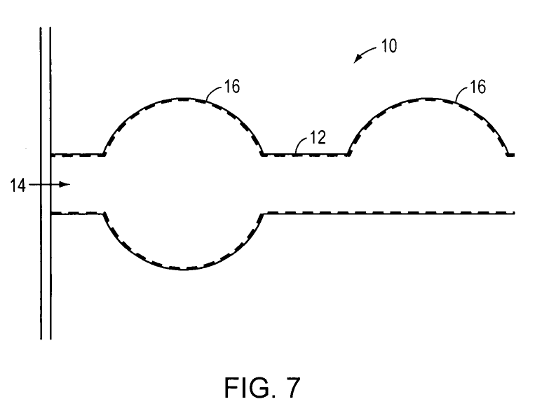

Controlling the size, shape, and positioning of an outpocketing permits regulation of the resulting fluid dynamics in and about the endovascular device. For example, the outpocketing can be designed to have a depth sufficient to create a recirculation zone in the lumen. In the endovascular device depicted in FIG. 7, more than one outpocketing 16 is incorporated, permitting the spatial varying of flow-related transport phenomena along the length of endovascular device 10. The outpocketings 16 can be identical or different (e.g. of differing shape, depth, or length).

An outpocketing can alter the mass transport properties by modifying the effective boundary layer. Mass transport to a surface, or wall flux has been extensively studied. In a recent review by Tarbell, describing the mass transport in arteries, wall flux is given by Jₓ=Kₗ(C_{b}−Cₛ) where Kₗ is the mass transport coefficient, C_{b} is the bulk concentration of solute and Cₛ is the surface concentration (Tarbell (2003)Annu. Rev. Biomed. Eng. 5:79-118). In a growing mass transport boundary layer, such as that which would occur at a wall step, outpouching or slit, Kₗ=D/δ where D is the diffusion coefficient and δ is the boundary layer thickness (Basmadjian et al. (1997) Biomaterials 18:1511-1522). Encountering a wall perturbation of length L and shear rate Y, δ is given by (D×L/Y)̂1/3, revealing the sensitivity of wall transport on perturbation geometry. In such a case, the total wall flux scales as L̂2/3 (Tarbell, 2003).

By changing the local convective transport properties, an introduced outpocketing significantly alters local drug delivery or recruitment. Balakrishnan et al. (2005) show the importance of stent strut perturbations in altering local wall transport from drug eluting stents (see, e.g., FIG. 4 of Balakrishnan et al.). While this study focuses on a setting where the geometric perturbation is much smaller than the arterial dimension (stent strut thickness ˜0.1 mm vs. luminal diameter ˜1 mm) similar implications are found when the perturbations are of similar magnitude as the artery dimension (Rappitsch et al. (1996) J. Biomechanics 29(2):207-215, e.g. FIGS. 3A and 4 of Rappitsch et al.).

The shape of an outpocketing can significantly affect fluid dynamics and drug delivery or recruitment. Numerical modeling of mass transport in realistic arterial settings illustrates the dramatic impact of vessel curvature and axial perturbations in vessel radius (Kaazempur-Mofrad et al. (2001) Ann. Biomed. Eng. 29(2):121-7). When multiple species are considered, allowing for wall reaction, the impact of flow can be dramatically altered. In an autocatalytic enzymatic reaction, the wall concentration of the produced species is given by K_{w}C_{b}/(1−K_{w}/Kₗ) where K_{w} is the wall reaction rate (Basmadjian et al., 1997). In such a case, as the mass transport coefficient Kₗ approaches K_{w}, the wall concentration can be explosively amplified, as is evidenced by aneurismal thrombus growth. As shown by Basmadjian et al., conditions can be established where wall concentration of the product can increase, decrease, or remain relatively insensitive to changes in mass transport (see, e.g., FIG. 5 of Basmadjian el al.). Accordingly, local delivery can be controlled by tailoring the local wall geometry as well as the exposed surface and its reactivity. These considerations further extend to the recruitment of cellular species such as platelets and leukocytes to local wall perturbations (Kuharsky et al. (2001) Biophys. J. 80(3):1050-1074; Richter et al. (2004) J. Clin. Invest. 113(11):1607-14), where far more complex interactions involving specialized adhesion and activation take place (Sorensen (2002) “Computational Simulation of Platelet Transport, Activation, and Deposition,” Ph.D. Thesis, University of Pittsburgh, see, e.g., FIG. 35 of Sorenson).

### Partitions

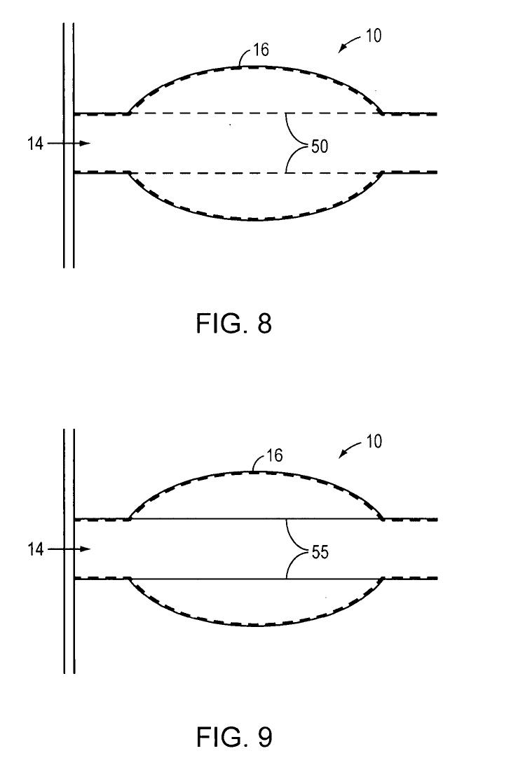

Local fluid dynamics can be further regulated by the addition of an optional partition between the outpocketing and the primary lumen through which blood flows through the vessel and the device. Endovascular devices incorporating such a partition are depicted in FIG. 8 and FIG. 9. Referring to FIG. 8, endovascular device 10 includes fenestrated partition 50 separating lumen 14 from outpocketing 16. Fenestrated partition 50 allows further control on transit time of bioactive agents to and from the partitioned luminal axial perturbations. Referring to FIG. 9, endovascular device 10 includes full partition 55 between lumen 14 and outpocketing 16. In the limit, all convective transport across the partition is stopped and transport takes place purely by diffusive processes determined by properties of the partition.

Partitioned outpocketings are readily implemented through various techniques. In one embodiment, the partition is a component of a unitary endovascular device. In such an embodiment, the partition is an elastic material that, following expansion, recoils to form a near tubular, minimal energy shape while the plastically deformable scaffold creates an outpocketing as illustrated in FIG. 9. In an alternative embodiment, the scaffold device is initially expanded to create the desired vessel contour. This initial expansion is followed by the sequential expansion of a second tubular device which implants the partition. In this second formulation, the partition need not necessarily be elastically deformable, but simply allow for plastic changes imposed by a second tubular stent structure in a manner paralleling the action of endovascular stent-grafts used to wall off pathologic aneurysms or dissections (Dake et al. (1999) J. Thorac. Cardiovasc. Surg. 116(5):689-703).

### Agents

The present invention is useful to regulate a local concentration of, for example, a bioactive agent released from the endovascular device; one or more agents recruited from the circulation; or an enzymatic reaction product by regulating delivery or recruitment in a manner dependent on flow rate. Depending on the overall geometry and flow characteristics, it is understood that the local concentration can be made to increase with flow, decrease with flow, or remain relatively constant regardless of flow.

Thus, in some embodiments, the endovascular device includes at least one bioactive agent, such as a drug, antibody, enzyme or small molecule. The bioactive agent, if present, can be releasably associated with the conformable scaffold or with the partition.

For example, the invention can be used to increase flow-mediated drug deposition to a luminal surface from a drug eluting stent. Furthermore, the pattern of deposition can be modulated by controlled variations in end-luminal vessel geometry. While certain profiles can lead to more homogenous distributions of drug at desirable concentrations, other profiles may promote axially or radially heterogeneous drug distributions of specified nature. In such fashion, particular loci of interest may be preferentially targeted. For example, anti-proliferative drug deposition may be preferentially deposited near the ends of a stent, which are typically recognized for their exaggerated, “candy-wrapper,” restenotic response. In other instances, drugs may be delivered preferentially to sites known to have endothelial denudation. In still other embodiments which employ the use of partitions, compounds, such as those promoting re-endothelialization, may be delivered by the partition itself. In some cases, subsets of agents may be delivered which counteract the adverse effects of the wall perturbation itself such as aneurismal thrombosis.

The technology may also be used to promote local recruitment of systemically circulating bioactive agents to the implantation site through altered transport phenomena (e.g. the recruitment of bioactive constituents in an aneurysm). These agents can be innate or exogenously introduced. The flow recruitment can occur in conjunction with local surface capture via the exposed stent surface. For example, specific cells can be trapped through the use of specific surface adherent molecules or antibodies. In some embodiments where a partition is used, the partition itself is used to promote recruitment of specific circulating agents.

This technology can also be used to promote local enzymatic reactions and to control the inflow and outflow of local substrate and product species. The enzymes can be locally delivered or bound to the device surface. Alternatively, they can be systemically circulating enzymes whose concentration is enhanced by the presence of flow perturbations (e.g. activated coagulation enzymes in an aneurysm). In one embodiment, formation of a mural thrombus within the outpocketing further serves to alter local wall transport. Such a strategy can be used, for example, to enhance wall uptake of drug released from the embedded scaffold, while helping to isolate the luminal surface from eluted drug, thus allowing bioactive agents, such as antiproliferative drugs, to minimize local restenosis, while enabling luminal growth of cells such as endothelial cells.

In some embodiments where a partition is used, the enzyme is associated with the partition. The substrates of these reactions can be locally released or systemically circulating. They can be innate or exogenously introduced. In one embodiment, the substrate species is an inactive drug (e.g. a prodrug) and the product species is the corresponding active drug. In another embodiment, the substrate is an inactive enzyme (e.g. a zymogen) and the product is an active enzyme.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are intended to be embraced therein.

What is claimed is:

1. A method comprising:
a) inserting into a blood vessel a conformable scaffold designed to deform a wall of the blood vessel;
b) radially expanding the scaffold, the expanded scaffold comprising a lumen permitting blood flow through the scaffold and blood vessel; and
c) controllably introducing at least one outpocketing in the scaffold and the wall of the blood vessel.

2. The method of claim 1, wherein steps b) and c) are performed concurrently.

3. The method of claim 1, wherein step b) comprises expanding a balloon within the lumen of the scaffold.

4. The method of claim 1, wherein the outpocketing creates a recirculation zone.

5. The method of claim 1, wherein the outpocketing is radially asymmetric.

6. The method of claim 1, wherein the outpocketing is radially symmetric.

7. The method of claim 1, wherein the scaffold comprises a bioactive agent releasably associated with the scaffold.

8. The method of claim 7, wherein the bioactive agent is a drug, antibody, enzyme, or small molecule.

9. An endovascular device comprising:
a conformable scaffold designed for insertion into a blood vessel and expansion within the blood vessel, the scaffold comprising one or more shaping elements forming, after expansion of the scaffold within the blood vessel, one or more outpocketings of predetermined shape in the scaffold and the wall of the blood vessel, the expanded scaffold comprising a lumen permitting blood flow through the scaffold and blood vessel.

10. The endovascular device of claim 9, wherein the one or more outpocketings create one or more recirculation zones.

11. The endovascular device of claim 9, wherein at least one shaping element comprises a shape-memory material that expands to form at least one outpocketing of predetermined shape.

12. The endovascular device of claim 9, wherein at least one shaping element comprises struts or wires of nonuniform thickness.

13. The endovascular device of claim 9, wherein an outpocketing is radially asymmetric.

14. The endovascular device of claim 9, wherein an outpocketing is radially symmetric.

15. The endovascular device of claim 9, further comprising a bioactive agent releasably associated with the scaffold.

16. The endovascular device of claim 15, wherein the bioactive agent is a drug, antibody, enzyme, or small molecule.

17. An endovascular device comprising:
a conformable scaffold designed for insertion into a blood vessel and expansion within the blood vessel, the scaffold comprising, after expansion within the blood vessel, (i) a lumen permitting blood flow through the scaffold and blood vessel and (ii) one or more outpocketings of predetermined shape;
the device further comprising a partition between an outpocketing and the lumen, wherein the partition is not apposed to the scaffold in the outpocketing, thereby defining a space between the outpocketing and the partition.

18. The endovascular device of claim 17, wherein the partition is formed of a material that is more elastic than the conformable scaffold.

19. The endovascular device of claim 17, wherein the partition creates a barrier to convective blood flow into and out of the space.

20. The endovascular device of claim 17, wherein the partition creates a barrier to diffusion into and out of the space.

21. The endovascular device of claim 17, the device comprising a bioactive agent releasably associated with the scaffold or with a surface of the partition that is not in contact with the lumen.

22. The endovascular device of claim 21, the device further comprising a second bioactive agent releasably associated with a surface of the partition that is in contact with the lumen.

23. The endovascular device of claim 21, wherein a surface of the partition that is in contact with the lumen comprises one or more molecules that bind endothelial cells or endothelial precursor cells.

24. The endovascular device of claim 23, wherein the one or more molecules comprise an extracellular matrix molecule.

25. A method of altering fluid flow in an endovascular device, the method comprising the step of:
inserting a partition into an expanded conformable scaffold in contact with an interior wall of a blood vessel, the scaffold having a lumen permitting blood flow through the scaffold and the blood vessel, the scaffold and the interior wall of the blood vessel comprising at least one outpocketing,
wherein the partition is positioned between the outpocketing and the lumen and is not apposed to the scaffold in the outpocketing, thereby defining a space between the outpocketing and the partition.

26. The method of claim 25, wherein the partition creates a barrier to convective blood flow into and out of the space.

27. The method of claim 25, wherein the partition creates a barrier to diffusion into and out of the space.

28. The method of claim 25, wherein a surface of the partition that comprises one or more molecules that bind endothelial cells or endothelial precursor cells is positioned to be in contact with the lumen.

29. The method of claim 25, further comprising the step of immobilizing the partition with respect to the endovascular device.

30. The method of claim 25, the method further comprising the step, after the step of inserting the partition into the expanded conformable scaffold, of expanding the partition within the conformable scaffold.

31. The method of claim 30, wherein the partition is plastically deformable.
